(19) United States
(12) Patent Application Publication
(10) Pub. No.: US 2006/0122535 A1
(43) Pub. Date: Jun. 8, 2006

(54) Method and device to obtain percutaneous tissue samples

(76) Inventor: Wolfgang Daum, Groton, MA (US)

Correspondence Address: Wolfgang Daum, 20 Whiley Road, Groton, MA, 01450, US

(21) Appl. No.: 11/297,710

(22) Filed: Dec. 8, 2005

Related U.S. Application Data
(60) Provisional application No. 60/634,386, filed on Dec. 8, 2004.

(51) Int. Cl. A61B 10/00

(52) U.S. Cl. 600/565; 600/567; 600/568

(57) ABSTRACT

A new method and design for a percutaneous biopsy system that cuts only the tissue lesion specimen and that does not penetrate through or beyond the targeted tissue into intact tissue. The proposed mechanism operates only in the targeted lesion space and leaves healthy or unsuspicious tissue intact. The proposed biopsy mechanism will cut the specimen in front of the tip of the guiding needle. The device may be image guided by ultrasound, any x-ray based modality or magnetic resonance (MRI).

### FIELD OF THE INVENTION

The present invention relates to a percutaneous biopsy device that cuts a suspicious desired tissue specimen, leaving undesired tissue unharmed. This application claims priority to U.S. Ser. No. 60/634,386, which incorporated herewith by reference.

### BACKGROUND OF THE INVENTION

Medical biopsy is a technique to obtain tissue samples for pathologic diagnostics. Open surgical biopsies are still the standard techniques in many medical fields. There are basically two principle percutaneous biopsy techniques for the interventional or minimally invasive biopsy market, which are fine needle aspiration and core biopsy. Approximately two million biopsies are performed in the United States each year.

The core biopsy technique—also known as Temno technique—is the oldest and most common biopsy technique on the market. Core biopsy devices are available as manually operated spring loaded or as fully automatic systems. All devices use a coaxial needle set consisting of an inner solid needle (obdurator) in which a little pocket (notch) is grinded and an outer hallow needle, which is beveled to have a sharp tip. The obdurator is pushed into the lesion and the surrounding tissue fills up the notch. Then, the hallow outer needle moves fast forward cutting the tissue to leave a sharp cut specimen in the notch. Core biopsy devices have a couple of disadvantages:

Bending: Due to the beveled tip of the obdurator and the thin notch strap, which makes the design unstable, the obdurator bends during its forward movement through the tissue towards the opposite side of the tip bevel. This bending makes the core biopsy devices imprecise in targeting smaller lesions.

Overshoot: Due to its mechanical design, core biopsy needles overshoot the targeted area by the length of the obdurator tip. A typically 18-gauge prostate biopsy device will overthrow the lesion by 5-10 millimeters and perforate the tissue on the distal other side of the lesion. Prostate cancer sites for instance are most likely been found in the peripheral zone of the prostate. Because the prostate has a diameter of minimum 3 to 5 centimeters, core biopsies are limited to the inner portion if one wants to leave the prostate attaching tissue intact. The same problem occurs, when targeted breast tumor lesions are close to the lung pleura. In brain tissue every needle penetration in healthy tissue may cause serious cognitive defects of the patient.

Artifact of needle tip under Magnetic Resonance Imaging (MRI): Due to the mechanical design of core biopsy needles, the tip of the obdurator needle is a solid piece of metal. Even if more MRI compatible material like titanium alloys are used for the material, this solid part causes a rather large artifact at the tip of the obdurator, especially when the needle is used in higher magnetic flux MRI tomographers, like 1.5 or 3.0 Tesla.

Half Volume Sample: Due to the notch pocket of the core biopsy needle, the sample volume is only half the diameter of the column shaped obdurator. While this is not considered a serious problem, it does lead to the use of larger sized biopsy needles to obtain the desired sample volume, while one could use a smaller needle size if the tissue sample where full column sized.

Torn Sample: While core biopsy devices work well in fatty tissue, they often have difficulties in more dense tissue. Here the cutting needle often rather tears the tissue resulting in an insufficient tissue sample for the pathological analysis.

Fine needle aspiration (FNA) biopsy techniques use a simple hallow needle, which is placed into the tissue. Vacuum to perform the aspiration is either applied by a vacuum pump or in the simplest form by an expanded syringe. Vacuum aspiration biopsy devices lack the disadvantage that they tear out the tissue and give imprecise specimen cuts. Further this type of biopsy can only be accomplished with softer and easier to tear tissue. Prostate, breast or brain tissue is not recommended for biopsy via aspiration.

The object of the here presented invention is to overcome above stated disadvantages of today's biopsy devices. The invention provides a biopsy mechanism which penetrated straight through the tissue without being bended, does not overshoot the tissue lesion, does not create an abnormal image artifact and precisely cuts the desired tissue piece as a full circle specimen.

### SUMMARY OF THE INVENTION

The suggested biopsy mechanism will cut the specimen in front of the tip of the guiding needle. The device may be guided by Magnetic Resonance Imaging (MRI), x-ray based techniques or ultrasound.

### BRIEF DESCRIPTION OF THE DRAWINGS

FIG. 1 illustrates the principle mechanism of the proposed biopsy principle.

FIG. 2 illustrates the difference in specimen gathered with
a.) conventional core biopsy and
b.) new proposed method, as described here.

FIG. 3 illustrated a possible driving mechanism for the biopsy device.

### DETAILED DESCRIPTION

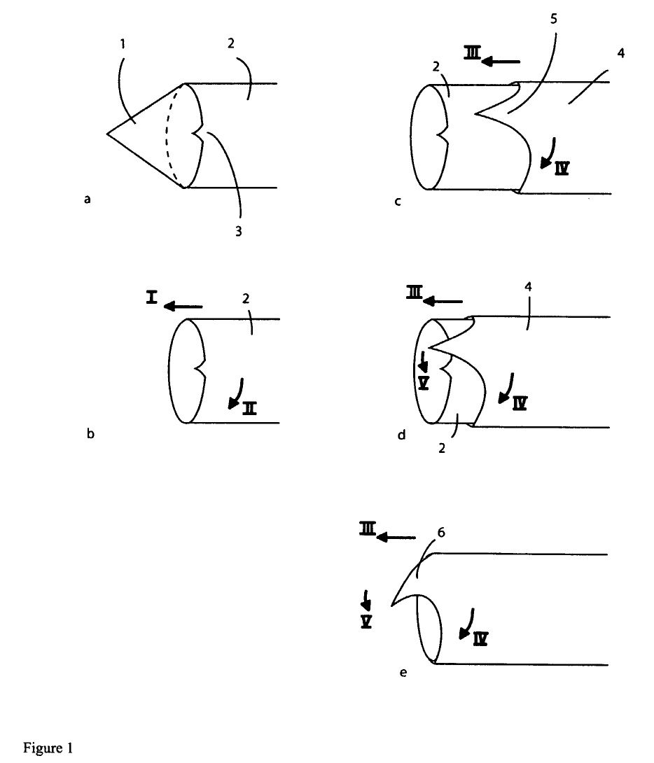

The new cutting mechanism cuts at the tip of a needle a dome like specimen. FIG. 1 illustrates the basic new biopsy concept operating in five time snap shot stages. Only the distally located parts of the instrument are shown; the proximal parts (handle) are not shown.

FIG. 1a shows the access tube 2 in which an inner stylet 1 with a trocar like tip is positioned. This needle set is percutaneously pushed through the patients tissue until the tip of the stylet 1 is positioned at the location of planned biopsy.

In FIG. 1b the inner stylet is withdrawn backwards and removed from the access tube 2. The access tube 2 now is rotated (arrow II) and pushed forward (arrow I) in distal direction. During this procedure tissue is cut by the cutting blade of the access tube 2 and a pillar like specimen collects within the access tube 2. The diameter of the specimen equals the inner diameter of the access tube 2, typically 1 mm to 4 mm. This collecting of specimen can be supported by aspirating vacuum with help of a pump or a syringe from the proximal side of the access tube 2.

FIGS. 1c to 1e now show how the specimen is planned to be cut at the distal end of the instrument.

In FIG. 1c a second tube, the cutting tube 4, is pushed from proximal to the distal direction over the access tube (arrow III). The cutting tube 4 comprises a cutting blade 5 at its tip and constantly rotates (arrow IV). The cutting blade 5 of the cutting tube 4 is pre-bend in such a way, that when overshooting the distal tip of the access tube 2—FIG. 1d—it bends back to its original form (arrow V). FIG. 1e shows the cutting blade 6 fully extended and bend back to its original form, which when rotated (arrow IV) is a dome like or half sphere form. The combination of forward-movement (arrow III) over the edge of the access tube tip and rotation (arrow IV) now cuts the dome like form from the tissue specimen, leaving a circular pillar of specimen in the access tube 2 with dome like circular tip.

The difference in specimen quality of a conventional core biopsy system and one as here proposed considering the same needle diameter and length is explained using FIG. 2. FIG. 2a illustrated a specimen of a conventional core biopsy needle. Due to the notch geometry the form of the gathered specimen is a half-circular pillar like. Because the tip of the obdurator needle penetrates through the biopsied lesion into the healthy tissue on the opposite side of the lesion, the tissue there is damaged, as illustrated in FIG. 2a with dashed lines. In opposite, the new proposed biopsy mechanism gives a true circular pillar like specimen with a calf circular dome on the distal top. The here new proposed biopsy mechanism will give more than 50% more pathological specimen, using the same needle diameter and length. Typical diameters and length of this mechanism are 1 mm to 3 mm in diameter and 10 mm to 12 mm in length, as pathologists are used to. The here new proposed biopsy mechanism leaves healthy tissue in tact.

The device may be made from stainless steel, Nivaflex®, titanium-vanadium-alloy, plastic, carbon fibre or nickel-titanium (NiTi). Typically the wall-thickness of any tube is between 0.01 millimetres and 0.5 millimetres. The access tube comprises a relative to its tube diameter small cutting blade. The cutting blade of the cutting tube may be made from different material as the cutting tube. The cutting blade of the cutting tube is pre-bend inwards to the centre of the cutting tube and its tip locates at the centre or beyond of the cutting tube. The cutting blade is welded or glued onto the cutting tube. The cutting blade is bending backwards when the cutting tube is sliding over the access tube. The movements of any tube are manually operated or motor driven. The gathered specimen has a typical diameter of 1 mm to 4 mm and length of 10 mm to 12 mm in length.

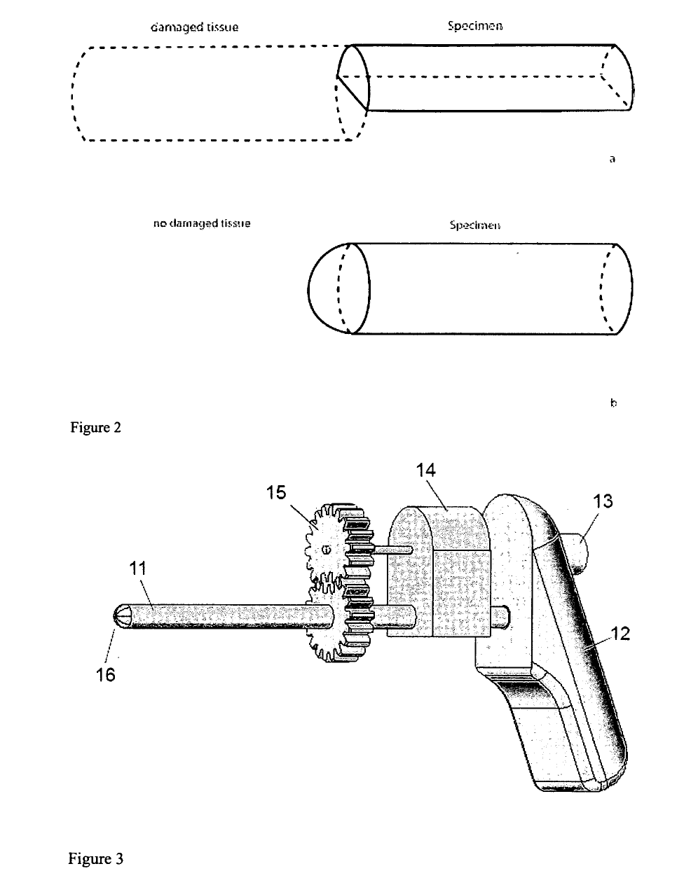

FIG. 3 illustrated a possible driving mechanism for the biopsy device in principle. The biopsy needle system 11 with rotating tip mechanism 16 is mounted in a hand held piece 12. A motor unit 14 generates the rotafion, which is transferred via a gear unit or transmission 15 to the needle system. A control mechanism (knob) 13 starts or stops the rotation.

What is claimed is:

1. A method to obtain tissue specimen using a device having an inner solid stylet with a bevelled tip, a hallow access tube and hallow cutting tube, whereas hallow cutting tube carries a pre-bend and inwards cutting blade on its tip, comprising the following procedural steps:
pushing forward the inner stylet through the tissue until its tip reaches the targeted lesion;
pushing forward the access tube sliding over the stylet until its tip reaches the targeted lesion;
pulling back the inner stylet;
rotating and pushing forward the cutting tube over and around the access tube until the cutting blade is fully bended to its unbend position.

2. The method of claim 1, wherein the forward speed of the access tube is between 0.1 millimetres per second and 100 millimetres per second.

3. The method of claim 1, wherein the forward speed of the cutting tube is between 0.1 millimetres per second and 100 millimetres per second.

4. The method of claim 1, wherein the rotation speed of the cutting tube is between 0.1 rounds per second and 10,000 rounds per second.

5. The method of claim 1, wherein tissue is drawn into the access tube by applying vacuum pressure to the proximal side of the access tube.

6. The method of claim 1, wherein the device is guided by one or any combination of the group of imaging methods consisting of x-ray fluoroscopy, computer tomography, magnetic resonance, ultrasound, visual, positron emission tomography or single photon emission computed tomography.

7. A device to obtain tissue specimen, comprising an inner solid stylet with bevelled tip, a hallow access tube and hallow cutting tube, whereas hallow cutting tube carries a pre-bend and inwards cutting blade on its tip.

8. The device of claim 7, whereas any tube, any part of a tube or the stylet is made from stainless steel, Nivaflex®, titanium-vanadium-alloy, plastic, carbon fibre or nickel-titanium.

9. The device of claim 7, whereas any wall-thickness of any tube is between 0.01 millimetres and 0.5 millimetres.

10. The device of claim 7, whereas the access tube comprises a relative to its tube diameter small cutting blade.

11. The device of claim 7, whereas the cutting blade of the cutting tube is of different material as the cutting tube.

12. The device of claim 7, whereas the cutting blade of the cutting tube is pre-bend inwards to the centre of the cutting tube and its tip locates at the centre or beyond of the cutting tube.

13. The device of claim 7, whereas the cutting blade is welded or glued onto the cutting tube.

14. The device of claim 7, whereas the cutting blade is bend backwards when the cutting tube is sliding over the access tube.

13. The device of claim 7, whereas the movements of any tube is manually operated.

14. The device of claim 7, whereas the movements of any tube is motor driven.

15. The device of claim 7, wherein the gathered specimen has a typical diameter of 1 mm to 4 mm and length of 10 mm to 12 mm in length
